(19) United States
(12) Patent Application Publication
(10) Pub. No.: US 2021/0295984 A1
(43) Pub. Date: Sep. 23, 2021

(54) OPTIMIZED PATIENT SCHEDULES BASED ON PATIENT WORKFLOW AND RESOURCE AVAILABILITY

(71) Applicant: KONINKLIJKE PHILIPS N.V., EINDHOVEN (NL)

(72) Inventors: Michael Prokle, Boston, MA (US); Ranjith Naveen Tellis, Tewksbury, MA (US); Sandeep Madhukar Dalal, Winchester, MA (US); Ushanandini Raghavan, Lexington, MA (US); Christopher Stephen Hall, Kirkland, NY (US); Yuechen Qian, Lexington, MA (US)

(21) Appl. No.: 17/261,699

(22) PCT Filed: Jul. 21, 2019
(86) PCT No.: PCT/EP2019/069608

§ 371 Date: Jan. 20, 2021

Related U.S. Application Data
(60) Provisional application No. 62/829,062, filed on Apr. 4, 2019.
(60) Provisional application No. 62/700,930, filed on Jul. 20, 2018.

(51) Int. Cl. G16H 40/20; G06Q 10/06; G06N 7/00

(52) U.S. Cl. CPC G16H 40/20; G06N 7/005; G06Q 10/06393; G06Q 10/0633

(57) ABSTRACT

Abstract: A non-transitory computer-readable medium stores instructions readable and executable by at least one electronic processor (20) to perform a workflow schedule monitoring method (100). The method includes: simulating (42) a workflow schedule (46) using data including workflow timestamps and a planned schedule; detecting (44) non-compliance of the workflow schedule with constraint data (52); in response to the detection of non-compliance, determining one or more workflow schedule adjustment options (48) for adjusting the workflow schedule to comply with the constraint data; and controlling a display device (24) of the workstation to display the workflow schedule and the one or more workflow schedule adjustment options.

### FIELD

The following relates generally to the radiation treatment arts, radiology arts, radiation planning arts, adaptive radiation treatment plan arts, and related arts.

### BACKGROUND

Hospital departments suffer from high variability in their workflow process. Most hospital department plan their day or several days in advance and schedule patients according to best practice, experience and scheduling algorithms. This planned schedule can include fixed appointments for outpatients and flexible time slots allocated for inpatients. Additional open time slots are allocated for emergency patients arriving last minute. Each patient group has different characteristics and requirements. Emergency patients have little to no flexibility in their arrival, outpatients expect to be serviced at their scheduled time and inpatients may be flexible over the day but also have other commitments over their stay in the hospital.

A given day may evolve significantly different from the original planned workflow schedule. Examples of unanticipated changes or variability in the workflow schedule include: early, late or no-show outpatients; delayed arrival of inpatients due to longer-than-anticipated transportation time from another hospital department; unpredictable number and timing of emergency patients; reduced staff availability due to staff illnesses, etc.; patient-to-patient variations in the actual time to perform a procedure (e.g., complications that extend a procedure); availability of equipment or rooms (e.g., limited number of available rooms & equipment or break-down of equipment), among others.

The process variability can lead to a variety of problems for hospital departments. Any delays in patient workflow schedule directly affect subsequent patients by delaying their appointment resulting in additional wait times. Similarly, staff members have to adapt to the workflow schedule change by increasing their working efficiency and/or working extended hours. Deviations from the planned workflow schedule directly affect patient and staff satisfaction leading to loss of hospital revenue (e.g., large amounts of unanticipated overtime can increase staff turnover, while excessive wait times are a common source of patient complaints).

At any given time, it may be difficult to predict how a given change will affect the future patient workflow schedules, associated resources, and how much the planned schedule deviates from what will actually occur. Taking a sick staff member as an example, it is difficult for the hospital to estimate how much the missing staff member will delay each of the patient appointments over the particular day and what corrective action (e.g., cancel one appointment or inform patients to arrive at a later point of time) to implement in order to minimize impact on patients, minimize impact on overhead costs, or otherwise minimize impact on key performance indicators (KPIs).

Referring physicians diagnosing patients sometimes require an imaging exam of the patient for better diagnosis. These imaging orders are typically entered into the computerized provider order entry (CPOE) system by the referring physician. The schedulers then pick these orders to schedule them based on ‘priority’ of the order and ‘order entered’ date. Outpatients receive a phone call to determine and schedule a suitable appointment time. Inpatients are more flexible in their appointment time and usually have predefined time slots reserved. Emergency patients receive highest priority over the other two patient type and extra capacity may be kept throughout the day.

During the scheduling process, it is very difficult to estimate the impact of the allocated appointment time on the overall performance of the workflow (e.g., How does this time slot affect the overall patient wait time? Does this appointment time balance staff and resource utilization?).

The following discloses new and improved systems and methods to overcome these problems.

### SUMMARY

In one disclosed aspect, a non-transitory computer-readable medium stores instructions readable and executable by at least one electronic processor to perform a medical workflow schedule monitoring method. The method includes: simulating a workflow schedule of medical examinations or medical therapy sessions using data including workflow timestamps and a planned schedule; detecting non-compliance of the workflow schedule with constraint data; in response to the detection of non-compliance, determining one or more workflow schedule adjustment options for adjusting the workflow schedule to comply with the constraint data; and controlling a display device of the workstation to display the workflow schedule and the one or more workflow schedule adjustment options.

In another disclosed aspect, a medical examinations or medical therapies workflow scheduling system includes a display device and one or more user inputs devices. At least one electronic processor of a computing device is programmed to: simulate a plurality of proposed workflow schedules of medical examinations or medical therapy sessions using data including workflow timestamps and a planned schedule; compute key performance indicators (KPIs) for the proposed workflow schedules; select one of the proposed workflow schedules based on the computed KPIs; control the display device to display the selected proposed simulated workflow schedule; and update one or more appointment time slots of the simulated workflow schedule with the selected by one of: (i) a manual confirmation input via the one or more user input devices or (ii) automatically updating the one or more appointment time slots of the simulated workflow schedule.

In another disclosed aspect, a medical examinations or medical therapies workflow scheduling method includes: receiving at least one medical examination or therapy session request to be scheduled; simulating a plurality of proposed workflow schedules of medical examinations or medical therapy sessions using data including workflow timestamps and a planned schedule for different selected schedule slots of the at least one medical examination or therapy session request to be scheduled, the simulating including mapping a probabilistic time evolution of states of the proposed workflow schedules as a function of time from an initial workflow schedule with a Bellman equation; computing key performance indicators (KPIs) for the proposed workflow schedules; selecting one of the proposed workflow schedules based on the computed KPIs; and controlling a display device to display the selected proposed simulated workflow schedule.

One advantage resides in reducing wait times for patients.

Another advantage resides in generating more efficient workflow schedules for medical laboratories.

Another advantage resides in increased medical staff and patient satisfaction.

Another advantage resides in predicting changes in future patient workflow schedules, associated resources, and costs.

Another advantage resides in real-time predictions of changes to a daily medical staff workflow schedule.

Another advantage resides in providing a scheduling device that reduces user effort in adjusting the schedule to remediate unanticipated events.

Another advantage resides in providing a user interface to visualize future patient appointments and necessary information.

Another advantage resides in generating data-driven customized patient appointment time slots.

Another advantage resides in providing a scheduling algorithm with a clinical department's specific workflow.

Another advantage resides in prioritizing patient procedures and appointments.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a workflow schedule monitoring system according to one aspect.

FIG. 2 shows exemplary flow chart operations of the system of FIG. 1.

FIG. 3 diagrammatically shows an illustrative workflow schedule depicted as a Gantt chart.

FIG. 4 diagrammatically discloses a scheduling learning engine of the system of FIG. 1.

FIG. 5 shows an exemplary list of an availability capacity and constraints on appointment types and restrictions on orders to be scheduled by the system of FIG. 1.

FIG. 6 shows an exemplary list of orders to be scheduled by the system of FIG. 1.

FIGS. 7 and 8 show example simulated workflow schedules generated by the system of FIG. 1.

FIGS. 9A-E show KPI results for various patient schedules generated by the system of FIG. 1.

FIG. 10 shows an overall KPI score for a patient schedule generated by the system of FIG. 1.

FIG. 11 shows another exemplary flow chart operation of the system of FIG. 1.

### DETAILED DESCRIPTION

In existing radiology lab or other medical laboratory settings, it is typical to rely upon a daily schedule of patients to coordinate workflow schedules over the day. This can lead to problems if patients arrive late, if laboratory personnel call in sick, if an imaging system or other laboratory equipment goes down, or other unanticipated events occur.

The disclosed approach employs a computer or other electronic processor programmed to provide a combination of a workflow schedule simulator, a workflow schedule optimizer, and a user interface (e.g. in conjunction with a display and a keyboard, mouse, touch-sensitive display, or the like) to provide proactive management of the daily schedule. A commercially available package such as FlexSim™ simulation software (available at https://healthcare.flexsim.com/) can be used to create a digital model of a planned workflow and simulate “what-if” scenarios. One or more potential schedules can be created and tested as “what-if” scenarios on the FlexSim™ simulation software. The simulation also takes into account available situational awareness information such as medical personnel availability based on whether they have clocked in for work, more finely grained locational information provided by a Real Time Locating Service (RTLS), location of outpatients via GPS (when available and authorized by the patient), status of imaging systems obtained from the Radiology Information System (RIS), and/or so forth.

The workflow schedule optimizer can be embodied as an add-on package (e.g., OptTek-OptQuest™, available at https://www.opttek.com) to the simulator, and operates to adjust aspects of the simulated workflow schedule in accordance with a set of business constraints/restrictions/priorities in order to generate schedule adjustments. For example, if a laboratory worker calls in sick, the simulator may estimate that this will lead to afternoon patients being delayed by delay times that accumulate over the course of the day. The workflow schedule optimizer then may simulate hypothetical workflow schedules for various candidate adjustments or combinations of adjustments, such as shifting times of adjustable patient appointments (e.g. in-patients), cancelling one or more patients, adding a temporary worker, contacting remote personnel to help in maintaining a workflow schedule, providing overtime to laboratory personnel in order to extend the work day, and/or so forth. Each such hypothetical simulation can be scored using one or more Key Performance Indicators (KPIs). The system may automatically choose one or more adjustments scoring highest in terms of KPIs, or may propose the highest scoring adjustment(s) to laboratory personnel via the user interface for user selection.

Implementation of selected adjustment(s) may be manual, semi-automated, or fully automated depending upon the type of adjustment, the desired level of human supervisory oversight, and available ancillary implementation systems. For example, rescheduling of an outpatient may be done manually, or may be done automatically via a robotic telephone call or texting system. Implementation of paid overtime may be implemented automatically or may require supervisory approval. In general, the daily schedule is not updated for an adjustment until confirmation of implementation of the adjustment is received by the system. The user interface may also provide an up-to-date workflow schedule in the form of a Gantt chart or other visualization.

The disclosed system is principally intended as a mechanism to improve daily scheduling on a time horizon of the remaining work day (or work shift). However, adjustments to the work schedule over the course of each day may be logged to generate a database of unanticipated events and work schedule adjustments made in response to those events. Such a database may be useful information for consideration by a Radiology Department manager in allocating departmental resources and/or advocating for increased departmental resources. In some examples, the disclosed system can be implemented in a hospital setting as a centralized system which monitors, forecasts, and optimizes workflow in the entire hospital.

It is not atypical for a hospital to have hundreds of outstanding medical imaging study orders at a given time. Presently, this is handled by manual scheduling, but this does not produce highly efficient schedules. In embodiments disclosed herein, a schedule learning engine performs Monte Carlo simulation of possible schedules. The workflow simulator operates to statistically simulate each such schedule configuration and KPIs for the configuration. A weighted combination of the KPIs may be employed as an objective function (or “score”) for assessing the schedule configurations. Some suitable KPIs include staff utilization, room utilization, total wait time, last patient exit-elapsed time (corresponding to the total length of the imaging work shift), and so forth.

In some embodiments disclosed herein, the schedule learning engine chooses the highest scoring Monte Carlo-simulated schedule configuration. In another possible approach, the schedule learning engine presents the top-N scoring Monte Carlo-simulated schedule configurations to the user on a display (e.g. a “dashboard”) for selection. In one practical implementation, such Monte Carlo simulations may be performed for various schedule slots for a single imaging examination in order to generate the top-N possible slots for that imaging examination. This could be displayed on the dashboard for the human scheduling agent, who can consult with the patient (or patient's representative) as to which of these N possible slots is preferred. A difficulty in the foregoing approach is that the number of Monte Carlo-simulated schedule configurations is limited by computational speed, especially when being run to assist a human scheduling agent in (near) real-time.

In other embodiments disclosed herein, the schedule learning engine employs reinforcement learning (e.g. 0-learning or Policy Gradient optimization) using a Bellman equation to map the time evolution of states as a function of time starting from some initial schedule. The reinforcement learning is trained on the Monte Carlo-simulated schedule configurations to select slots with the best long-term payoff. Reinforcement learning advantageously exploits a certain payoff and at the same time explores newer actions (slot selections) to prevent it from always greedily selecting the next slot with decent payoff. Hence, the reinforcement learning has particular advantages for the medical imaging study scheduling task at hand.

The imaging study orders which are scheduled by the schedule learning engine are suitably input as a list of orders. Fields may be provided to indicate study priority, medical imaging procedure (from which can be derived the imaging modality and hence the imaging rooms that can perform the procedure), and patient class (e.g., in-patient or out-patient).

In a further variant, the workflow simulation may incorporate a prediction model for patient no-shows and cancellations. Patient appointment preferences may also be incorporated, both individual (specific patient X cannot be examined the week of the 20th) and statistical (outpatients prefer morning appointments).

The disclosed schedule learning engine may be utilized in various ways. In one approach, as discussed above the scheduler may be applied to work through the list of orders one-by-one, possibly in conjunction with a human scheduling agent viewing a dashboard who makes the final schedule slot determinations. In another approach (not mutually exclusive), the schedule learning engine can be accessed by the patient directly via a mobile application (“app”) that presents the dashboard, and the patient can schedule (or reschedule) his or her own medical imaging study appointment using the schedule learning engine.

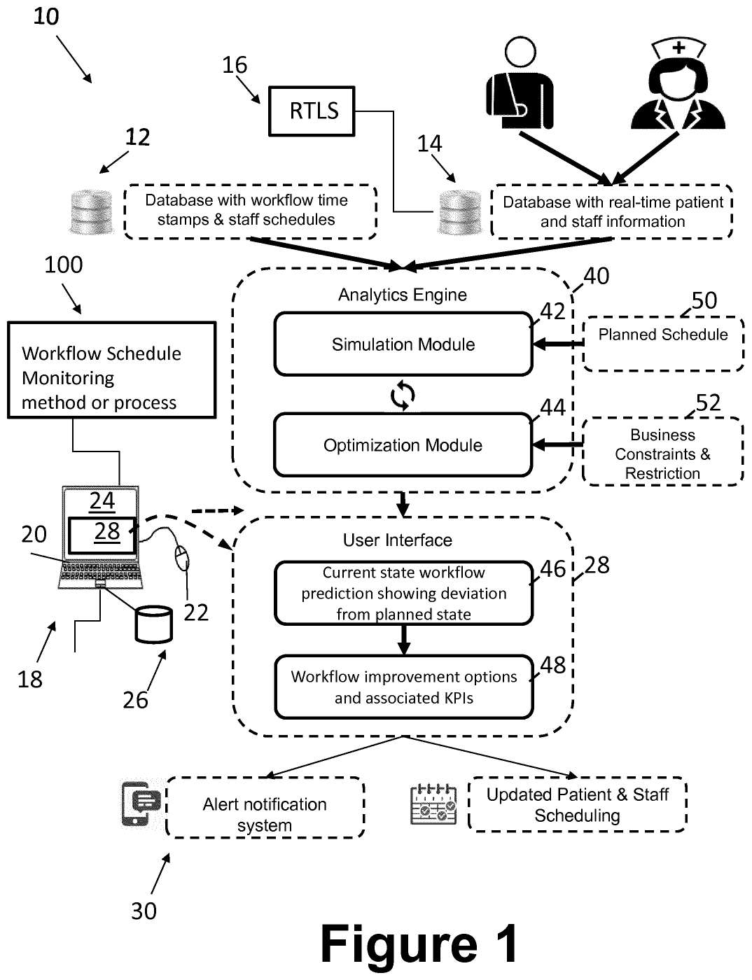

With reference to FIG. 1, an illustrative workflow schedule monitoring system 10 is shown. As shown in FIG. 1, the system 10 includes a first database 12, a second database 14, a real-time location service (RTLS) device 16, and a computing device 18 (e.g., a workstation, a computer, a tablet, a smartphone, and so forth). The first database 12 is configured to store “past” information such as workflow schedule process time stamps, staffing schedules and clinical resource availability. In some example, the first database 12 can be an electronic medical record (EMR) database. The second database 14 is configured to store “present” information such as real-time patient and staff locations (e.g., via GPS data), along with real-time environmental information (e.g., weather data, traffic data, and so forth). The RTLS device 16 generates position data of the staff and patients (and optionally also mobile medical equipment that may be assigned to the laboratory on an occasional basis), and stores this data in the second database 14. By way of non-limiting illustration, one example of a suitable RTLS is an RFID-based RTLS employing radio frequency identification (RFID) tags worn by staff, on a patient bracelet, disposed on or in tracked equipment, or so forth and tracked by RFID tag readers placed at strategic locations around the hospital or other medical facility. In another example, an RFID tag can be worn by a staff member or the patient (e.g., on a wristband, an article of clothing, an identification badge), or placed in an area where the staff member or patient is typically found (e.g., in a car or home) to allow for remote location monitoring of the patient or staff member. An RTLS tags database stores tag-subject assignments enabling association of RFID tags with the tagged individuals or equipment, and an electronic map of the hospital or other medical facility (or a surrounding area thereof) identifies the location based on which RFID tag reader picks up the RFID tag (or, in a more advanced embodiment, detection of the RFID tag by two or three RFID tag readers enables more precise location by way of triangulation).

In another non-limiting illustration, the RTLS 16 can employ a smartphone, a tablet, or another smart device operated by the staff member or the patient. In this example, the user can log-in into a mobile application (“app”) on their smartphone or tablet, and use the global positioning system (GPS) in the phone or tablet to collect position information and determine a location of the staff member or patient. The computing device 18 at the medical facility can then use the determined location from the RTLS 16 and generate a route for the staff member or patient to arrive at the hospital, which can be displayed on the smartphone or tablet.

For the purposes of the workflow scheduling, it may be sufficient for the RTLS 16 to be used to classify each patient or staff member as one of (1) not in the hospital; (2) in the hospital but not at the radiology lab; or (3) at the radiology lab. In the case of mobile medical equipment, typically only categories (2) or (3) will apply. In some embodiments, the RTLS 16 can be used to determine if a staff member is available. For example, if the location of each staff member is known, then the locations can be compared to the planned schedule to infer staff utilization (e.g., staff member A is scheduled for a procedure on patient B with staff member C). In another example, the location information can be used for historical timestamps (e.g., nurse A is utilized for X minute for procedure Y), which can be stored in the first database 12.

The workstation 18 comprises a computer or other electronic data processing device with typical components, such as at least one electronic processor 20, at least one user input device (e.g., a mouse, a keyboard, a trackball, and/or the like) 22, and a display device 24. It should be noted that these components can be variously distributed. For example, the electronic processor 20 may include a local processor of a workstation terminal and the processor of a server computer that is accessed by the workstation terminal. In some embodiments, the display device 24 can be a separate component from the computer 18. The workstation 18 can also include one or more databases or non-transitory storage media 26. The various non-transitory storage media 12, 14, 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth. They may also be variously combined, e.g. a single server RAID storage may store both databases 12, 14. The display device 24 is configured to display a graphical user interface (GUI) 28 including one or more fields to receive a user input from the user input device 22.

In some embodiments, the system 10 also includes an alert generation device 30 configured to generate an alert based on an adjustment of a proposed workflow schedule. For example, the alert generation device 30 can include a device to generate a Messaging Service (MS) text message, a Short Messaging Service (SMS), an alert in a web-based program such as Microsoft Outlook, and so forth in order to inform a patient of rescheduling of the patient's appointment time. In some embodiments the patient may be given the option to accept or reject the rescheduling, in which case the system will not update the schedule to reflect the rescheduling unless and until the patient accepts by way of a return text message.

The system 10 is configured to perform a workflow schedule monitoring method or process 100. A non-transitory storage medium stores instructions which are readable and executable by the at least one electronic processor 20 of the workstation 18 and to perform disclosed operations including performing the workflow schedule monitoring method or process 100. In some examples, the methods 100 and/or 200 may be performed at least in part by cloud processing. The instructions which are executed to perform the workflow schedule monitoring method or process 100 may be viewed as implementing: (i) an analytics engine 40 including a workflow schedule simulation module 42 and a workflow schedule optimization module 44, and (ii) the user interface 28, e.g. controlling the workstation 18 to display on the display 24 a current workflow schedule 46 (i.e. the workflow schedule 46 in its current state as output by the analytics engine 42) and proposed workflow schedule adjustment options 48 for improving the workflow schedule, which are currently proposed but not yet implemented into the current workflow schedule 46 (for example, because the proposed adjustment options 48 have not been accepted or approved by the user, or because a proposed rescheduling of a patient has not been confirmed by the patient, hospital ward, or other authorizing entity, or so forth). At the beginning of the day the current workflow schedule may be set to a planned schedule 50, which is updated throughout the day by way of acceptance of proposed adjustment options 48 generated by the optimization module 44 of the analytics module 42.

In optimizing the workflow schedule, the optimization module 44 uses one or more key performance indicators (KPIs) as metrics of the quality of the optimized schedule. By way of non-limiting illustrative example, the KPIs may, for example, include one or more of: total predicted patient waiting time for all patients scheduled for procedures; maximum waiting time predicted for any single patient scheduled for a procedure (e.g., if patients A, B, C, D, and E have respective predicted waiting times of 2 min, 5 min, 25 min, 7 min, and 4 min, then the maximum waiting time KPI value would be 25 min); total operating costs; staff costs; total staff overtime; performance of the computing device 18; in-constraint status of the system; and/or so forth. These illustrative KPIs are each preferably minimized, but the optimization can alternatively be formulated as a maximization problem. The optimization figure of merit (i.e. objective function) can include a weighted combination of several KPIs, with weighting values chosen to scale the values to comparable units (e.g., time-based KPIs and cost-based KPIs are made comparable by suitable scaling) and to weight the relative importance of the various KPIs.

The optimization module 44 may perform a constrained optimization in which certain business constraints or restrictions 52 must be met by the optimized workflow schedule. By way of non-limiting illustrative example, the business constraints or restrictions may include one or more of: maximum waiting time predicted for any single patient, (this could be both a KPI to be minimized and a constraint if some maximum permissible waiting time for any patient is specified, e.g., at a patient service level in which the wait time should be less than or equal to 15 minutes); maximum number of hours worked by any staff member; maximum total staff overtime; maximum number of patient procedures per day; a constraint that no single patient can have more than one procedure; and/or so forth.

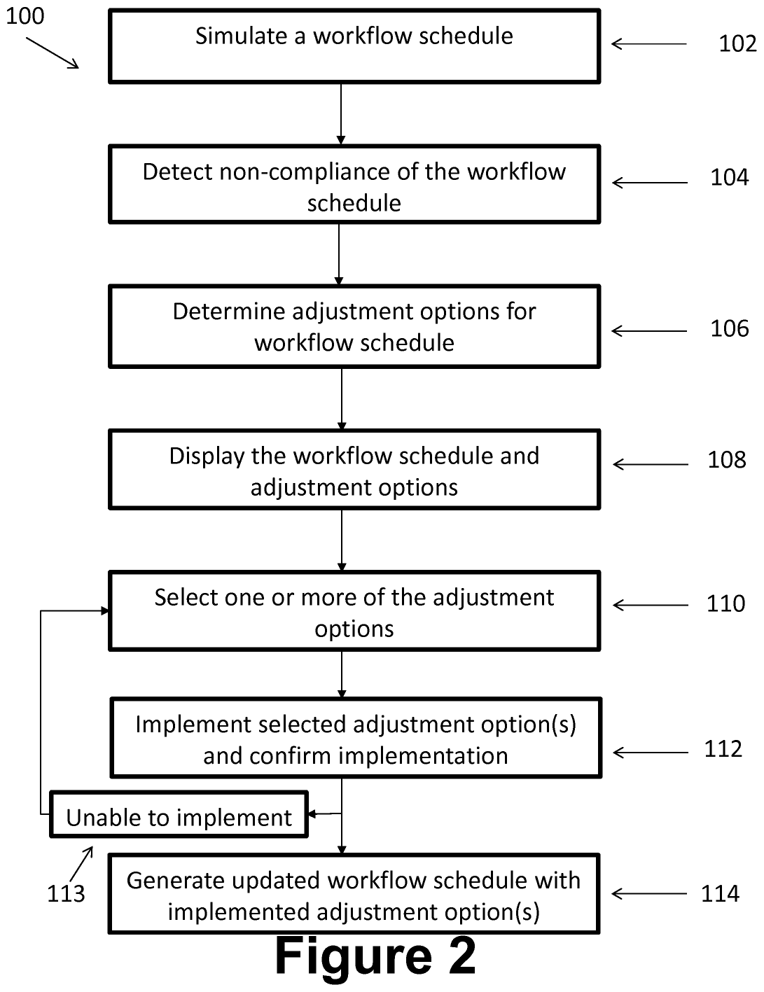

With reference to FIG. 2, an illustrative embodiment of the workflow schedule monitoring method 100 is diagrammatically shown as a flowchart. At 102 (e.g. performed by the simulation module 42 in the illustrative logical module architecture of FIG. 1), the at least one electronic processor 20 is programmed to simulate a workflow schedule using data including at least one of workflow timestamps, staff schedules, real-time patient location information, real-time staff location information, real-time staff location weather information, real-time staff location traffic information, and a planned schedule. For example, the workflow timestamps and the staff schedules can be retrieved from the first database 12, and the real-time patient location information and the real-time staff location information can be retrieved from the second database 14. The simulation operation includes updating and using the latest process distributions for workflow schedule simulations over a time period that allows statistically significant conclusions. In some examples, this process can be performed with manual time stamps by hospital staff, time stamps stored in the first database 12, or information provided by the RTLS 16. Since the hospital environment is constantly changing (e.g., a physician is getting quicker in performing a procedure), the timestamps allow to use the latest distributions that are statistically significant to use. In other examples, the timestamp data can be used in future scheduling operations (e.g., the hospital schedules more emergency patients in future weeks). The simulation operation simulates the planned schedule, as well as “what-if” scenarios using a set of latest recorded time stamps and an estimated patient arrival time. In some examples, the simulation includes generating key performance indicators (KPIs) (e.g., patient wait time, last patient existing, and so forth) for each appointment in the planned schedule. In one illustrative embodiment, the simulation module 42 is implemented as FlexSim™ simulation software suitably configured with the foregoing information and linked to appropriate available data sources (e.g. the databases 12, 14, the RTLS 16, or so forth).

At 104, the at least one electronic processor 20 is programmed to optimize the proposed workflow schedule (e.g., performed by the optimization module 44 of FIG. 1). To do so, the at least one electronic processor 20 is programmed to detect non-compliance at 104 of the workflow schedule with the constraint data 52 including, for example, staff hours, patient appointment times, and a maximum remaining number of patient appointments. Note that the constraints may be time-dependent and may change as the day progresses. For example, if 20 Magnetic Resonance Imaging (MRI) sessions are schedule per day, then at the beginning of the day, this optimization limit will be 20. On the other hand, when the optimization is run during the workflow schedule, for example after lunch, then this limit may be 10 remaining MRI sessions. In some examples, the detecting operations includes predicting a late arrival or absence of a patient or hospital staff member based at least on the real-time patient location information or the real-time staff location information and rerunning the simulating incorporating the predicted late arrival to detect the non-compliance of the workflow schedule with constraint data.

At 106, the at least one electronic processor 20 is programmed to, in response to the detection of non-compliance, determine one or more workflow schedule adjustment options for adjusting the workflow schedule to comply with the constraint data. The adjustment options can include any suitable adjustment to remove deviations from the workflow schedule. In one example, the adjustment option can include bringing in an additional hospital staff person (e.g. a staff member already in the medical facility or a staff member working at another, remote location in a hospital network). In another example, the adjustment option can include rescheduling a patient appointment. Each candidate adjustment is analyzed by invoking the simulation module 42 to simulate the workflow schedule with that adjustment, and the KPIs are computed for the resulting simulated workflow schedule to assign a score for that workflow schedule and for the corresponding candidate adjustment. By way of illustration, consider a situation where at 104 it is detected that the number of patients remaining on the schedule (say, 7 patients) is higher than the maximum allowable number of patients at the present time (say, 6). This may occur, for example, if one or more imaging procedures ran longer than anticipated, so that the time remaining in the workday is insufficient to provide service to all 7 remaining patients. Then the candidate adjustments may include: removal of a first of the remaining 7 patients and simulating that workflow schedule; removal of a second of the remaining 7 patients and simulating that workflow schedule; and so forth until the option of removing each of the 7 patients is simulated. The KPIs are computed for each simulated workflow schedule and the options are ranked by the scores. In some examples, the KPIs can be used to determine tradeoffs between resources (e.g., staff overtime costs, patient wait time costs, etc.) to make scheduling decisions.

By way of a second illustration, consider a situation where one staff member becomes sick or has a family emergency, and must leave at noon. At 102 the workflow schedule with that staff member now removed is simulated, and at 104 it is detected that with this change the constraint data 52 that a patient/staff ratio of 4:1 is maintained. There may be several options that can overcome non-compliance with this 4:1 patient/staff ratio constraint. One option may be for a patient to be rescheduled for another day. Another option may be for an additional staff member to be brought in. A third option may be for a current staff member to agree to work overtime. A fourth option may include rerouting a staff member at another medical facility location in the hospital network or at not at the hospital altogether, and using the RTLS 16 (e.g., an RFID tag in an identification badge of the staff member or attached to the staff member's clothing; tracking the staff member via the GPS in their smartphone or tablet; and the like) to plan a route or reroute the staff member from the other facility to the hospital. Each such option is evaluated at 106 by invoking the simulation module 42 to simulate the workflow schedule with that option implemented, and the option is scored by computing the KPIs for the simulated workflow schedule. The options are then ranked by the computed KPI-based scores.

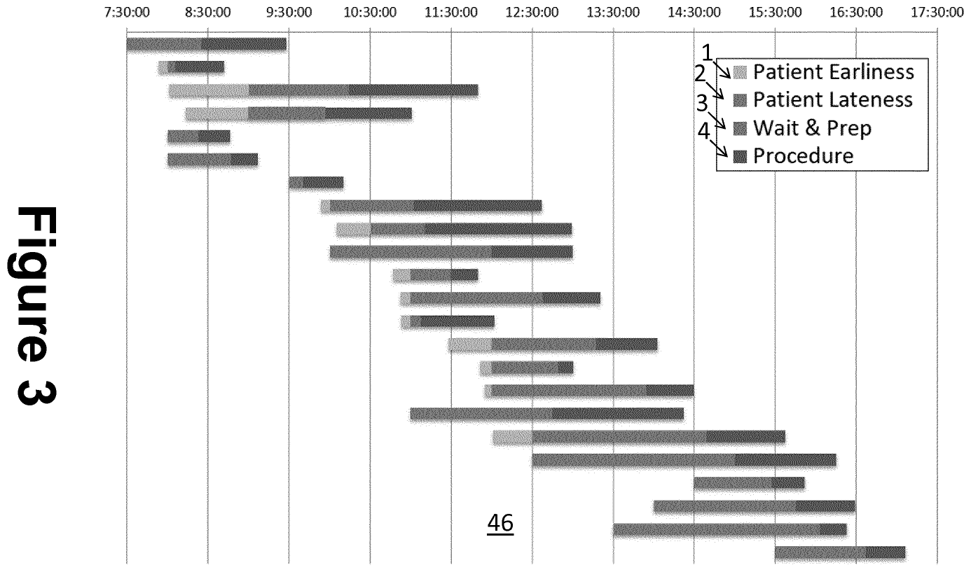

At 108, the at least one electronic processor 20 is programmed to control the display device 24 to display the workflow schedule computed at 102 and the one or more workflow schedule adjustment options developed at 106, preferably as a ranked list (ranked by their KPI scores) and optionally listed with those scores. In some embodiments only the top-N ranked options may be listed, e.g. only the two or three top-scoring options. The workflow schedule 46 and the adjustment options 48 can be displayed via the GUI 28 as diagrammatically indicated in FIG. 1. In one example, the workflow schedule 46 is displayed as a Gantt chart (see FIG. 3, where each horizontal bar corresponding to a patient; although not shown in FIG. 3, each horizontal bar is contemplated to be labeled appropriately, e.g. by patient name, type of imaging procedure, and/or so forth). The use of a Gantt chart for displaying the workflow schedule 46 advantageously enables immediate visual recognition for any given time (horizontal axis) of how many patients are predicted to be undergoing service (indicated by how many horizontal bars cross that time) and what stage of procedure each patient is predicted to be in at that time (using color coding or other distinctive coding of portions of the horizontal bar representing the patient). The displayed workflow schedule 46 can show the planned workflow and highlight the deviations therefrom. In some examples, the at least one electronic processor 20 is programmed to control the display device 24 to display the associated KPIs associated with each option (generated at 102).

At 110, the at least one electronic processor 20 is programmed to receive, via the one or more user inputs devices 22, user inputs indicative of selection of one of the workflow schedule adjustment options. This corresponds to an operation of the diagrammatically illustrated GUI 28 of FIG. 1. For example, the user can select one or more of the displayed adjustment options (e.g., request additional staff, reschedule an appointment, and so forth).

At 112, the selected adjustment option(s) are implemented. This may be done manually, semi-automatically, or fully automatically depending upon the option being implemented, the desired level of human supervisory oversight (if any), and the available implementation infrastructure. For example, if the option to be implemented is a rescheduling of an outpatient's appointment, the implementation 112 may comprise activating the alert notification system 30 of FIG. 1 to send a text message to the outpatient requesting to reschedule, and receiving a return text message from the outpatient approving rescheduling. On the other hand, if the selected adjustment option is to have a staff member work overtime, then this may be implemented automatically or, in a variant embodiment, a request for overtime authorization may be sent to an appropriate hospital official and the option deemed implemented upon receipt of such authorization. In the case of rescheduling an in-patient, implementation may entail connecting with a Hospital Information System (HIS) or other database and automatically updating the patient's schedule in the HIS to reflect the rescheduling. These are merely non-limiting illustrative examples.

In some instances, the selected adjustment option may not be able to be implemented, as indicated in FIG. 2 at 113. For example, an outpatient may not respond to the text message requesting rescheduling and hospital policy may be that an appointment cannot be rescheduled without contacting the patient; or, a staff overtime request may not be denied by the appropriate hospital official, or so forth. In such a case, the selected option which cannot be implemented is removed from the list of available options and flow passes back to 110 to present the remaining option(s), preferably with some displayed explanation that the originally selected option was not implemented.

At 114, in the opposite case in which the selected adjustment option is successfully implemented, the at least one electronic processor 20 is programmed to generate an updated workflow schedule by adjusting the workflow schedule in accord with the selected workflow schedule adjustment option. For example, when one or more of the displayed adjustment options are selected, the displayed workflow schedule can be updated and displayed based on the selected options. In some examples, the deviations between the actual workflow and workflow schedule change on the display device 24 based on the selected and implemented adjustment options. The at least one electronic processor 20 is then programmed to control the display device 24 to display the updated workflow schedule. In some examples, the at least one electronic processor 20 is programmed to store, in the second database 14, the selected workflow schedule adjustment options used to update the displayed schedule. In some embodiments, the simulation, detecting, and options determination operations (e.g., 102-106) can be repeated upon receiving, via the one or more user inputs devices 22, one or more user inputs indicative of selection of one or more of the displayed workflow schedule adjustment options.

FIG. 3 shows an example of the workflow schedule 46 as a Gantt chart. Each horizontal bar corresponds to a patient. Each color shade (labelled 1-4) is indicative of a different component of the report (e.g., patient earliness, patient lateness, wait and preparation time, and procedure). Although not shown in FIG. 3, each horizontal bar is contemplated to be labeled appropriately, e.g. by patient name, type of imaging procedure, and/or so forth.

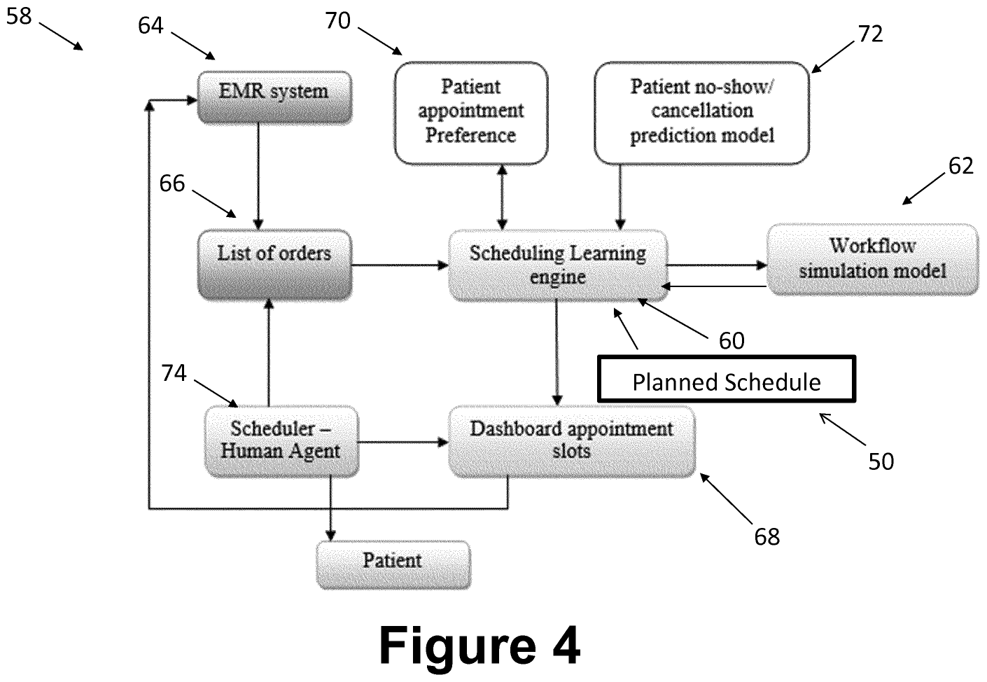

FIG. 4 shows an example embodiment of a scheduling assistant 58 of the system 10 to assist the user in generating the planned schedule 50. A scheduling learning engine 60 is configured to generate a workflow simulation model 62 which simulates the actual workflow. The model 62 captures all the tasks patients flow through including the process time (as a distribution) for each task, the resources necessary to perform the task like a CT room, portable ultrasound equipment, a nurse, a physician etc. The model 62 also captures the number of available resources and their schedules. By passing the patients appointment time and their procedure type to the model, the scheduling learning engine 60 can compute the KPIs like the patient wait/idle time, arrival to exit time, last patient exit time, staff/room/equipment utilization etc. This module can be developed using discrete event simulation or agent-based simulation techniques.

The scheduling learning engine 60 is operatively connected with an EMR system 64 that contains a list of orders 66. The scheduling learning engine 60 is configured to retrieve the list of orders 66 from the EMR system 64.

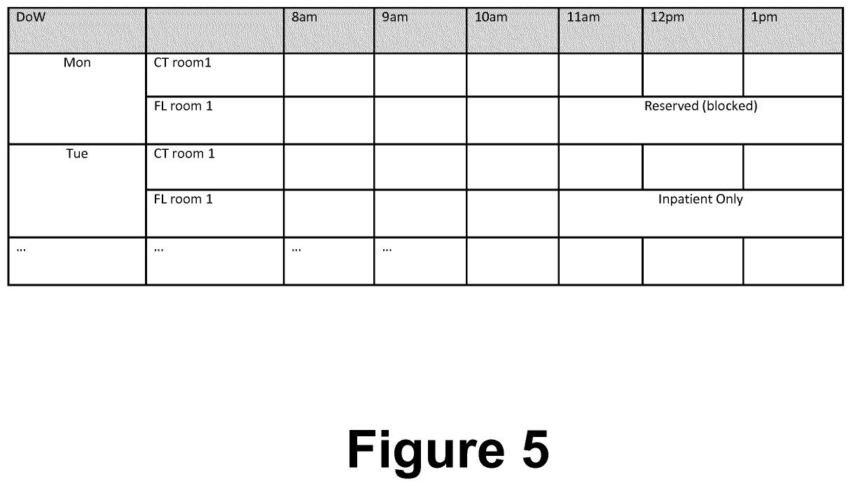

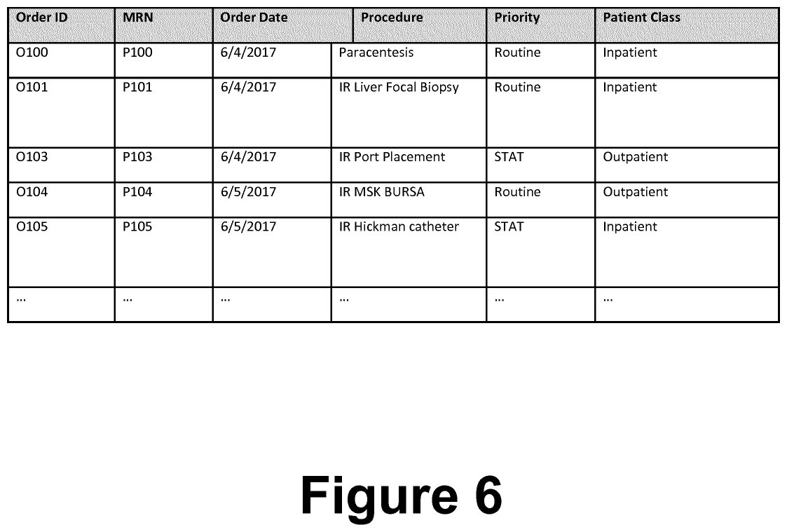

The scheduling learning engine 60 is configured to identify optimized patient schedules that have been tested on the model of the workflow. An initial state of the patient schedule (i.e., the current planned schedule 50) is shown as depicted in FIG. 1. The planned schedule 50 shown in FIG. 5 shows an availability capacity and constraints on appointment types and restrictions on orders to be schedule (e.g., the vacant slots and some reserved for Inpatient and some that are blocked). The patient orders to be scheduled can be retrieved from the EMR system 64 which contains information like the order created date, procedure type etc. as shown in FIG. 6 as an exemplary list of orders to be scheduled by the system of FIG. 1.

Referring back to FIG. 4, the scheduling learning engine 60 iteratively simulates an action of randomly assigning an order that is to be scheduled to an appointment time. Placing an order in an empty slot in adherence to patient preference, positively rewards the system and any violations, for example placing an Outpatient order which was reserved for Inpatient will result in a negative reward. Any such rules can be coded into the reward system. The slot duration estimates to perform a certain imaging procedure like a “Liver Biopsy” can be a random draw from the probability distribution of the curated historical data which the workflow model 62 can provide. Several patient schedules can be generated using Monte Carlo sampling techniques.

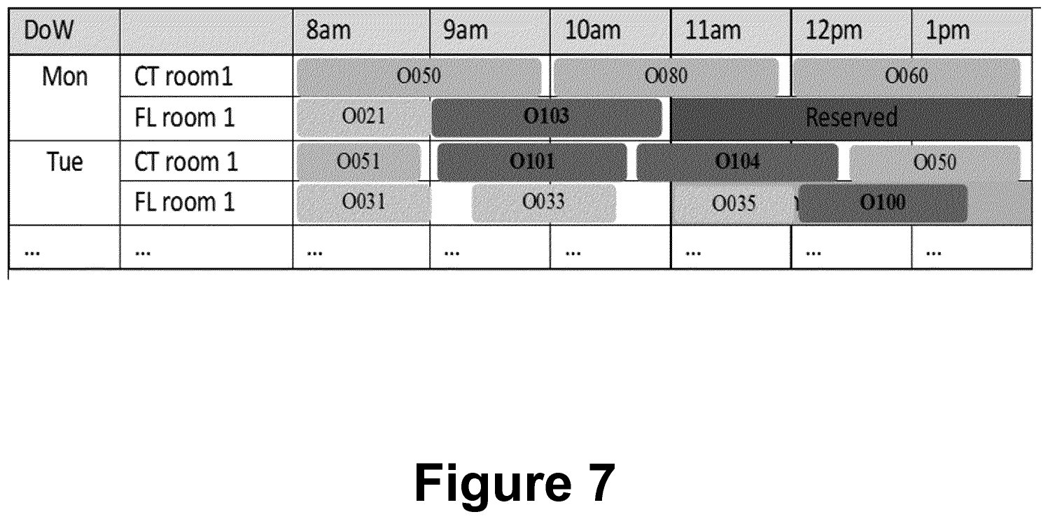

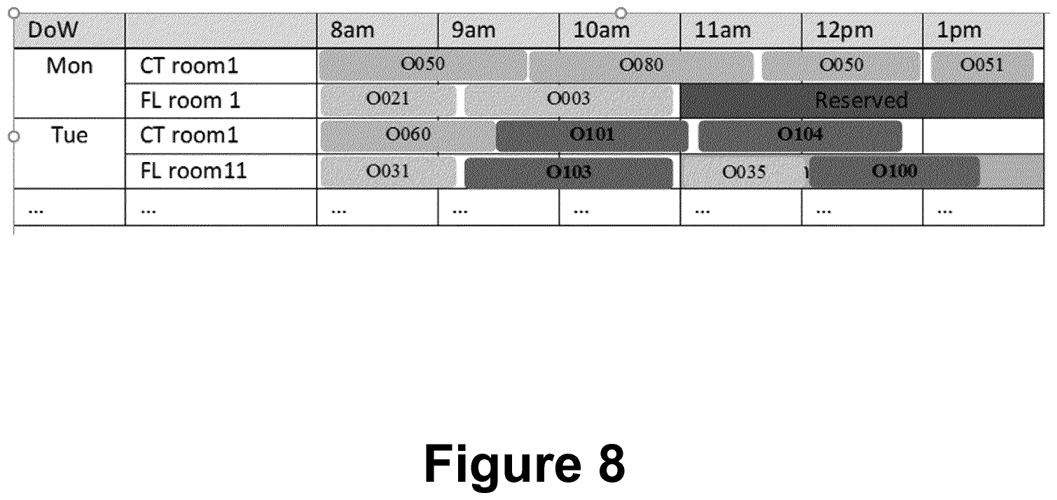

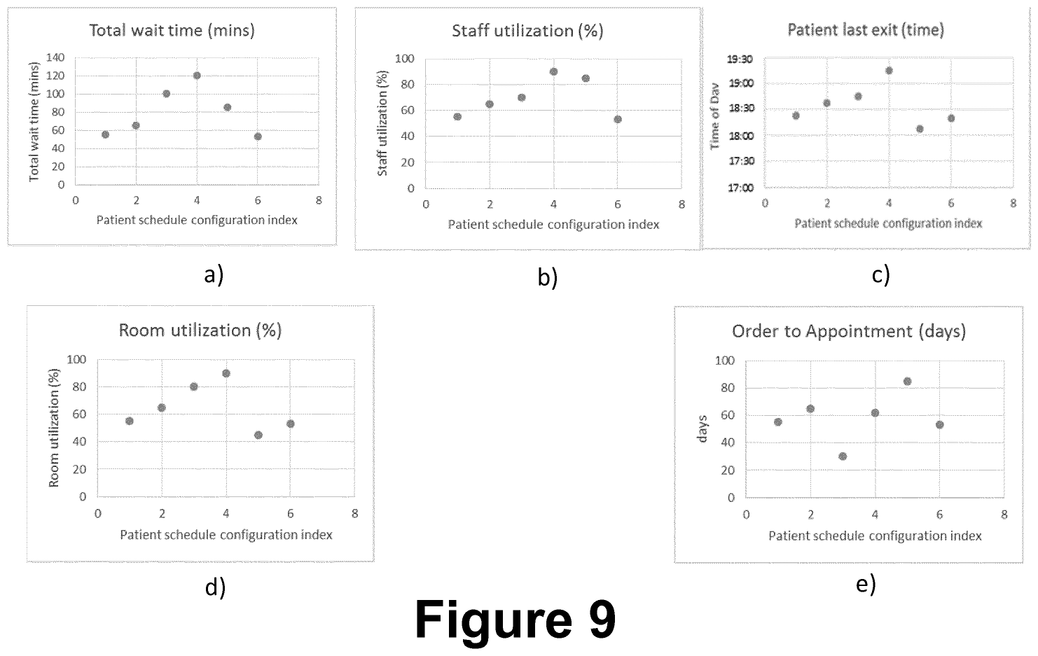

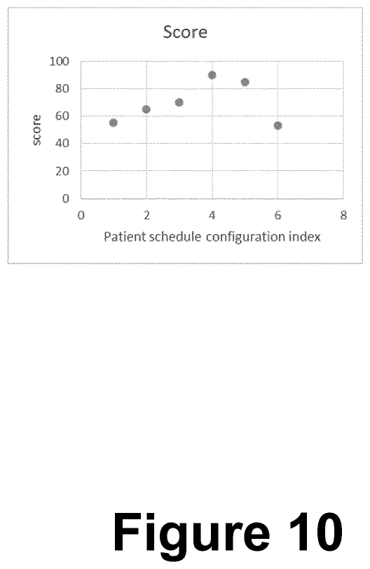

These test patient schedules generated as shown in FIG. 7 and FIG. 8, are passed as inputs to the workflow model 62. The workflow model 62 executes and outputs the KPIs like the total patient wait time, staff and room utilization etc. that can be expected for the given patient schedule. These KPI values can be further translated into a reward or value function that the system can use, to learn the outcome of its actions and maximize the cumulative future reward. This process can continue for a fixed number of iterations or until an objective function is optimized. The overall performance of the various patient schedules is illustrated in FIGS. 9A-E. It shows the output of the KPI for each patient schedule configuration. A final score can be computed by combining all the KPI values to determine the overall best patient schedule configuration as shown in FIG. 10. For example, a simple arithmetic score can have all the positively correlated KPIs values in the numerator and the negatively correlated ones in the denominator. Score=(Staff+Room Utilization)/(Total wait time+Last patient exit elapsed time). The final appointment slots are now contiguous in time without the concept of a fixed slot size.

The combinations of appointment times to immediate and long-term rewards/value mapping can be represented by the Bellman equation. Such a learning agent can be built using Reinforcement learning algorithms like the Q-learning or Policy Gradient approaches. The agent learns to pick an action with the best long-term payoff. The algorithms are capable of exploiting a certain payoff and at the same time explore newer actions to prevent it from being greedy.

Referring back to FIG. 4, the scheduling learning engine 60 is operatively connected with a scheduling dashboard 68 which can, for example, be displayed on the display device 24 of FIG. 1. The scheduling dashboard 68 is provided with a few suggested appointment slots for each patient order which are provided by the scheduling learning engine 60, along with the impact on the overall performance KPIs also provided by the scheduling learning engine 60; but there is always a possibility that the patient may request for a change. The scheduling dashboard 68 then can provide these options to the patient (or to a user of the scheduling system 10, e.g., a clerical staff person who maintains/updates the planned schedule 50 with the assistance of the scheduling assistant 58) to choose from and confirm the appointment time. The impact of any change request can easily be identified by testing the new schedule against the simulated workflow and the yet to be scheduled orders can be optimized to the newer state. Alternatively, the system 10 can send these options directly to the registered patient via a SMS or email to choose a convenient appointment time. The system 10 can update the effect on the KPIs as changes are made to the patient schedule.

The scheduling learning engine 60 can implement a patient appointment preference module 70 and/or a patient no-show/cancellation module 72. The patient appointment preferences can be collected from the patient during registration or inferred from past appointments. Examples for preferences could be appointments on weekdays or weekends, mornings or evenings etc. These preferences can be coded into the reward calculation system. Existing models predicting the probability of no-shows/cancellations if available can be modeled to test the impact on the KPIs and other appointments.

The scheduling learning engine 60 can also be operatively connected to a scheduling module 74 which can agent verify and choose the appropriate schedule and communicating with the patient to confirm the appointment. In a typical approach, the planned schedule 50 is not updated directly by the scheduling assistant 10, rather, the scheduling assistant 10 provided one or more suggested slots for an imaging examination order but the planned schedule 50 is not actually updated until receipt of a manual confirmation via human agent 74 the suggested slot. (In alternative embodiments, the scheduling assistant 10 does directly update the planned schedule 50, and if a user wishes to override the suggested slot the user then manually edits the automatically updated planned schedule). Alternatively, the system 10 can automatically communicate a few appointment options to the patient and confirm the booking. The scheduling module 74 views the list of orders and schedule them one by one.

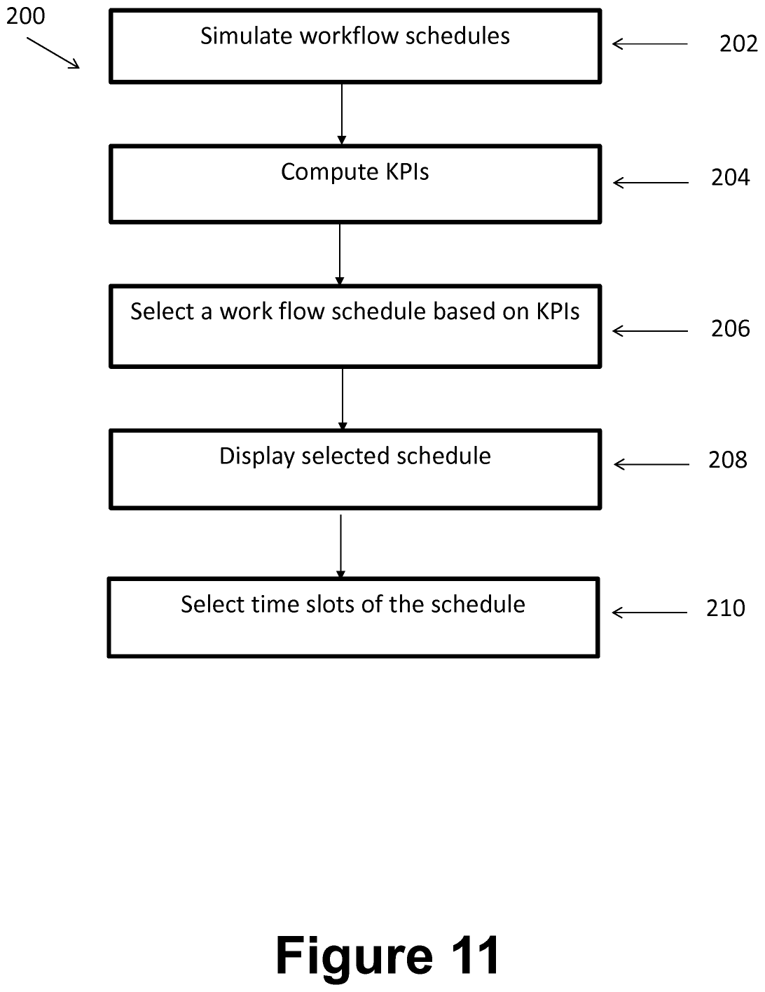

FIG. 11 shows an example of an illustrative embodiment of a medical examinations or medical therapies workflow schedule monitoring method 200 is diagrammatically shown as a flowchart. The method 200 can be executed by the at least one electronic processor 20 or the scheduling assistant 50. At 202, a plurality of proposed workflow schedules 46 of medical examinations or medical therapy sessions is simulated 42 using data including workflow timestamps and a planned schedule. Operation 202 can correspond to operation 102 of the method 100. For example, the plurality of proposed workflow schedules 46 are simulated by Monte Carlo simulation.

In some embodiments, at least one medical examination or therapy session request to be scheduled from one or more users is received by workflow schedule simulation module 42. The request from the users can be scheduling requests (preferred dates, time or day, and so forth). The plurality of proposed workflow schedules 46 are simulated for different selected schedule slots of the at least one medical examination or therapy session request to be scheduled. For example, the plurality of proposed workflow schedules 46 can be simulated with patient appointment preferences used in selecting the different selected schedule slots of the at least one medical examination or therapy session request to be scheduled. In other embodiments, the plurality of proposed workflow schedules 46 are simulated with a patient no-show and cancellation module. For example, the workflow schedule simulation module 42 performs simulations in which patients do not show up for an appointment (i.e., in real time). The workflow schedule simulation module 42 then adjusts the workflow schedule 46 to account for these missed appointments. In another example, the workflow schedule simulation module 42 performs simulations in which patients cancel appointments (i.e., in advance). The workflow schedule simulation module 42 then adjusts the workflow schedule 46 to account for these cancelled appointments.

In further embodiments, the plurality of proposed workflow schedules 46 are simulated by mapping a probabilistic time evolution of states of the proposed work schedules as a function of time from an initial work schedule. For example, the mapping of the probabilistic time evolution of states comprises mapping the probabilistic time evolution of states of the proposed work schedules with a Bellman equation.

At 204, KPIs are computed for the proposed workflow schedules 46. The KPIs are used to optimize the workflow schedules 46. In optimizing the workflow schedule, the optimization module 44 uses one or more KPIs as metrics of the quality of the optimized schedule. By way of non-limiting illustrative example, the KPIs may, for example, include one or more of: total predicted patient waiting time for all patients scheduled for procedures; maximum waiting time predicted for any single patient scheduled for a procedure (e.g., if patients A, B, C, D, and E have respective predicted waiting times of 2 min, 5 min, 25 min, 7 min, and 4 min, then the maximum waiting time KPI value would be 25 min); total operating costs; staff costs; total staff overtime; performance of the computing device 18; in-constraint status of the system; staff utilization, room utilization, total patient wait time, and last patient exit elapsed time; and/or so forth. These illustrative KPIs are each preferably minimized, but the optimization can alternatively be formulated as a maximization problem. The optimization figure of merit (i.e. objective function) can include a weighted combination of several KPIs, with weighting values chosen to scale the values to comparable units (e.g., time-based KPIs and cost-based KPIs are made comparable by suitable scaling) and to weight the relative importance of the various KPIs.

At 206, one of the proposed workflow schedules 46 is selected based on the computed KPIs. In one embodiment, the KPIs are summed for each of the proposed work schedules 46 to generate an overall KPI score for each proposed work schedule. The proposed workflow schedule 46 having the highest overall KPI score is selected. In another embodiment, the display device 24 can display the plurality of workflow schedule 46 having higher overall KPI scores relative to the proposed workflow schedules that are not selected.

At 208, the display device 24 is controlled by the at least one electronic processor 20 to display the selected proposed simulated workflow schedule 46. At 210, user inputs are received (via the one or more user input devices 22) indicative of a selection one or more time slots of the displayed workflow schedules 46. In another example, the display device 24 is controlled to display user input fields editable with the one or more user input devices 22, user input fields including study priority, medical imaging procedure, and patient class.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1-15. (canceled)

16. A medical examinations or medical therapies workflow scheduling system, comprising:
a display device;
one or more user inputs devices; and
at least one electronic processor of a computing device programmed to:
receive at least one medical examination or therapy session request to be scheduled:
simulate a plurality of proposed workflow schedules of medical examinations or medical therapy sessions using data including workflow timestamps and a planned schedule for different selected schedule slots of the at least, one medical examination or therapy session request to be scheduled, wherein the plurality of proposed workflow schedules are simulated by mapping a probabilistic time evolution of states of the proposed workflow schedules as a function of time from an initial workflow schedule, and wherein the mapping of the probabilistic time evolution of states comprises mapping the probabilistic time evolution of states of the proposed workflow schedules with a Bellman equation;
compute key performance indicators (KPIs) for the proposed workflow schedules;
compute key performance indicators (KPIs) for the proposed workflow schedules;
select one of the proposed workflow schedules based on the computed KPIs, wherein the KIPIs include one or more of staff utilization room utilization total patient wait time, and last patient exit elapsed time
control the display device to display the selected proposed simulated workflow schedule; and
update one or more appointment time slots of the simulated workflow schedule with the selected by one of: (i) a manual confirmation input via the one or more user input devices or (ii) automatically updating the one or more appointment time slots of the simulated workflow schedule.

17. (canceled)

18. (canceled)

19. The system of claim 16, wherein the at least one electronic processor is further programmed to:
sum the KPIs for each of the proposed workflow schedules to generate an overall KPI score for each proposed workflow schedule;
select the proposed workflow schedule having a highest overall KPI score.

20. The system of claim 16, wherein the at least one electronic processor is further programmed to:
sum the KPIs for each of the proposed workflow schedules to generate an overall KPI score for each proposed workflow schedule;
control the display device to display a plurality of the proposed workflow schedules having a higher overall KPI scores relative to the proposed workflow schedules that are not selected.

21. The system of claim 20, wherein the at least one electronic processor (14) is further programmed to:
receive, via the one or more user inputs devices, user inputs indicative of a selection of one of the displayed workflow schedules.

22. The system of claim 16, wherein the plurality of proposed workflow schedules are simulated by Monte Carlo simulation.

23. (canceled)

24. (canceled)

25. The system of claim 16, wherein the at least one electronic processor is further programmed to:
control the display device to display user input fields editable with the one or more user input devices, user input fields including study priority, medical imaging procedure, and patient class.

26. The system of claim 16, wherein the at least one electronic processor is further programmed to:
simulate the plurality of proposed workflow schedules with patient appointment preferences used in selecting the different selected schedule slots of the at least one medical examination or therapy session request to be scheduled.

27. The system of claim 16, wherein the at least one electronic processor is further programmed to:
simulate the plurality of proposed workflow schedules with a patient no-show and cancellation model.

28. A medical examinations or medical therapies workflow scheduling method, comprising:
receiving at least one medical examination or therapy session request to be scheduled;
simulating a plurality of proposed workflow schedules t of medical examinations or medical therapy sessions using data including workflow timestamps and a planned schedule for different selected schedule slots of the at least one medical examination or therapy session request to be scheduled, the simulating including mapping a probabilistic time evolution of states of the proposed workflow schedules as a function of time from an initial workflow schedule with a Bellman equation;
computing key performance indicators (KPIs) for the proposed workflow schedules, wherein the KPIs Include one or more of: staff utilization, room utilization, total patient wait time, and last patient exit elapsed time;
selecting one of the proposed workflow schedules based on the computed KPIs; and
controlling a display device to display the selected proposed simulated workflow schedule; and
updating one or more appointment time slots of the simulated workflow schedule with the selected by one of: (i) a manual confirmation input via the one or more user input devices or (ii) automatically updating the one or more appointment, time slots of the simulated workflow schedule.

29. (canceled)

30. The method of claim 28, further including:
summing the KPIs for each of the proposed workflow schedules to generate an overall KPI score for each proposed workflow schedule;
selecting the proposed workflow schedule having a highest overall KPI score.

31. The method of claim 28, further including:
summing the KPIs for each of the proposed workflow schedules to generate an overall KPI score for each proposed workflow schedule;
controlling the display device to display a plurality of the proposed workflow schedules having a higher overall KPI scores relative to the proposed workflow schedules that are not selected.

32. The method of claim 31, further including:
receiving, via the one or more user inputs devices, user inputs indicative of a selection of one of the displayed workflow schedules.

33. The method of claim 28, wherein the plurality of proposed workflow schedules are simulated by Monte Carlo simulation.

34. The method of claim 28, further including:
controlling the display device to display user input fields editable with the one or more user input devices, user input fields including study priority, medical imaging procedure, and patient class.

35. The method of claim 28, further including:
simulating the plurality of proposed workflow schedules with patient appointment preferences used in selecting the different selected schedule slots of the at least one medical examination or therapy session request to be scheduled; and
simulating the plurality of proposed workflow schedules with a patient no-show and cancellation model.

36. A non-transitory computer-readable medium storing instructions readable and executable by at least one electronic processor to perform a workflow schedule monitoring method, the method comprising:
retrieving, from a database, data related to workflow timestamps and staff schedules;
simulating a workflow schedule of medical examinations or medical therapy sessions using data including the workflow timestamps, staff schedules, patient location information, and staff location information and a planned schedule;
detecting non-compliance of the workflow schedule with constraint data, wherein the constraint data includes maximum total staff hours and a maximum remaining number of patient appointments; and the detecting includes predicting a late arrival or absence of a patient or hospital staff member based at least on the real-time patient location information or the real-time staff location information and rerunning the simulating incorporating the predicted late arrival to detect the non-compliance of the workflow schedule with the constraint data;
in response to the detection of non-compliance, determining one or more workflow schedule adjustment options for adjusting the workflow schedule to comply with the constraint data; and
controlling a display device of the workstation to display the workflow schedule and the one or more workflow schedule adjustment options.

37. The non-transitory computer-readable medium of claim 16, wherein the patient location information is real-time patient location information and the staff location information is real-time staff location information.

38. The non-transitory computer-readable medium of claim 36, wherein the method further includes:
receiving, via one or more user inputs devices of the workstation, user inputs indicative of selection of one of the workflow schedule adjustment options.

39. The non-transitory computer-readable medium of claim 38, wherein the method further includes:
generating an updated workflow schedule by adjusting the workflow schedule in accord with the selected workflow schedule adjustment option; and
controlling the display device to display the updated workflow schedule.

40. The non-transitory computer-readable medium of claim 36, wherein the method further includes:
controlling the display device to display associated key performance indicators (KPIs) associated with each option.

41. The non-transitory computer-readable medium of claim 40, wherein the method further includes:
repeating the simulation, detecting, and options determination operations upon receiving, via one or more user inputs devices, one or more user inputs indicative of selection of one or more of the displayed workflow schedule adjustment options.

42. The non-transitory computer-readable medium of claim 40, wherein the KPIs include one or more of: total predicted patient waiting time for all patients scheduled for procedures; maximum waiting time predicted for any single patient scheduled for a; total operating costs; staff costs; total staff overtime; performance of the computing device; and in-constraint status of the system.

43. The non-transitory computer-readable medium of claim 38, wherein the method further includes:
responsive to the selected workflow adjustment option, generating an alert to summon the additional hospital staff person; and
sending the alert to one or more hospital staff members.

44. The non-transitory computer-readable medium of claim 38, wherein the method further includes:
responsive to the selected workflow adjustment option, generating a rescheduling alert; and
sending the rescheduling alert to one or more patients.

45. The non-transitory computer-readable medium of claim 36, wherein the method further includes:
retrieving, from a database, data related to real-time patient location information, real-time staff location weather information, and real-time staff location traffic information; and
performing the simulation operation using the retrieved data.
